(19) United States
(12) Patent Application Publication
(10) Pub. No.: US 2026/0237492 A1
(43) Pub. Date: Aug. 13, 2026

(54) SYSTEMS FOR SEGMENTING CORONARY ARTERIES AND PERICORONARY ADIPOSE TISSUE BASED ON SELF-SUPERVISED LEARNING

(71) Applicant: HARBIN MEDICAL UNIVERSITY, Harbin (CN)

(72) Inventors: Ye TIAN, Harbin (CN); Qing DONG, Harbin (CN); Ying LIU, Harbin (CN); Hongyu WANG, Harbin (CN); Shuyuan GUO, Harbin (CN)

(73) Assignee: HARBIN MEDICAL UNIVERSITY, Harbin (CN)

(21) Appl. No.: 19/632,393

(22) Filed: Mar. 29, 2026

Related U.S. Application Data
(63) Continuation of application No. PCT/CN2025/137628, filed on Nov. 25, 2025.

(30) Foreign Application Priority Data
Dec. 4, 2024 (CN) 202411772142.X

(51) Int. Cl. G16H 30/40; G06N 3/045; G06N 3/0895; G06T 7/10

(52) U.S. Cl. CPC G16H 30/40; G06N 3/045; G06N 3/0895; G06T 7/10; G06T 2207/10081; G06T 2207/20081; G06T 2207/20084; G06T 2207/30101

(57) ABSTRACT

Provided is a system for segmenting coronary arteries and pericoronary adipose tissue based on self-supervised learning. The system includes: acquiring a training set of CCTA images, preprocessing the acquired training set and an image to be segmented, extracting segmentation masks from labeled images, performing joint pre-training based on the preprocessed training set to obtain a pre-trained encoder, performing joint training based on the segmentation masks to obtain a trained encoder, performing coronary artery segmentation on the preprocessed image to be segmented based on the trained encoder and a coronary artery segmentation module to obtain a coronary artery segmentation result, and processing the coronary artery segmentation result to obtain an adipose tissue segmentation result.

### CROSS-REFERENCE TO RELATED APPLICATIONS

This application is a Continuation of International Application No. PCT/CN2025/137628, filed on Nov. 25, 2025, which claims priority to Chinese Patent Application No. 202411772142.X, filed on Dec. 4, 2024, the entire contents of each of which are hereby incorporated by reference.

### TECHNICAL FIELD

The present disclosure relates to the technical field of medical image analysis, and particularly relates to a system for segmenting coronary arteries and pericoronary adipose tissue based on self-supervised learning.

### BACKGROUND

Coronary computed tomography angiography (CCTA), as a non-invasive and information-rich diagnostic tool, has been widely used for the assessment of coronary artery lesions. The attenuation of pericoronary adipose tissue (PCAT) is considered a novel biomarker associated with coronary artery inflammation, and has been demonstrated to correlate with the severity of coronary artery lesions. PCAT attenuation evaluated through CCTA is becoming a predictive indicator of acute coronary syndrome. Therefore, reliable automated coronary artery segmentation not only facilitates anatomical assessment but also supports subsequent quantification of PCAT attenuation.

The development of deep learning has introduced a new paradigm for medical image segmentation. However, the dependence of supervised learning models on large-scale labeled datasets constitutes a major bottleneck, particularly in the case of CCTA images where professional annotations are scarce and costly. Self-supervised learning, as an emerging branch of unsupervised learning, extracts feature representations based on unlabeled data and subsequently performs task-specific fine-tuning based on a small amount of annotated data. Therefore, how to efficiently perform coronary artery and pericoronary adipose tissue segmentation based on self-supervised learning remains an urgent problem to be solved.

Therefore, it is necessary to provide a system for segmenting coronary arteries and pericoronary adipose tissue based on self-supervised learning, so as to solve the problem that existing supervised learning models rely on large-scale labeled datasets, and to reduce the training cost and usage cost of the model.

### SUMMARY

One or more embodiments of the present disclosure provide a system for segmenting coronary arteries and pericoronary adipose tissue based on self-supervised learning, comprising an acquisition module, a preprocessing module, an extraction module, an encoder, a reconstruction module, a contrast module, a coronary artery segmentation module, and an adipose tissue segmentation module; wherein: the acquisition module is configured to acquire a training set of coronary computed tomography angiography (CCTA) images, the training set including labeled images and unlabeled images; the preprocessing module is configured to preprocess the training set and preprocess an image to be segmented; the extraction module is configured to extract segmentation masks from the preprocessed labeled images; the reconstruction module, the contrast module, and the encoder are configured to perform joint pre-training based on the preprocessed training set to obtain a pre-trained encoder; the coronary artery segmentation module and the pre-trained encoder are configured to perform joint training based on the segmentation masks to obtain a trained encoder; the encoder and the coronary artery segmentation module are configured to perform coronary artery segmentation on the preprocessed image to be segmented to obtain a coronary artery segmentation result of the image to be segmented; and the adipose tissue segmentation module is configured to process the coronary artery segmentation result to obtain an adipose tissue segmentation result of the image to be segmented.

### BRIEF DESCRIPTION OF THE DRAWINGS

The present disclosure will be further elucidated by way of exemplary embodiments, which are described in detail with reference to the accompanying drawings. These embodiments are not limiting. In these embodiments, the same reference numerals denote the same structures, wherein:

FIG. 1 is a schematic diagram of modules of a system for segmenting coronary arteries and pericoronary adipose tissue based on self-supervised learning according to some embodiments of the present disclosure.

FIG. 2 is a schematic diagram illustrating a training process of the system for segmenting coronary arteries and pericoronary adipose tissue based on self-supervised learning according to some embodiments of the present disclosure.

FIG. 3 is a schematic structural diagram of an encoder and a reconstruction module according to some embodiments of the present disclosure.

FIG. 4 is a flowchart of an algorithm for adipose tissue segmentation according to some embodiments of the present disclosure.

### DETAILED DESCRIPTION

To more clearly illustrate the technical solutions of the embodiments of the present disclosure, the accompanying drawings to be used in the description of the embodiments will be briefly described below. Obviously, the accompanying drawings in the following description are only some examples or embodiments of the present disclosure, and that the present disclosure may be applied to other similar scenarios in accordance with these drawings without creative labor for those of ordinary skill in the art. Unless obviously acquired from the context or the context illustrates otherwise, the same numeral in the drawings refers to the same structure or operation.

It should be understood that “system,” “device,” “unit,” and/or “module” as used herein is a way to distinguish between different components, elements, parts, sections, or assemblies at different levels. However, these words may be replaced by other expressions if they accomplish the same purpose. The term “and/or”, as used herein, is merely a way of describing the associative relationship of an associated object, indicating that three relationships may exist, e.g., A and/or B, which may be represented as: A alone, both A and B, and B alone.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

Flowcharts are used in the present disclosure to illustrate the operations performed by the system according to some embodiments of the present disclosure. It should be understood that the operations described herein are not necessarily executed in a specific order. Instead, they may be executed in reverse order or simultaneously. Additionally, one or more other operations may be added to these processes, or one or more operations may be removed.

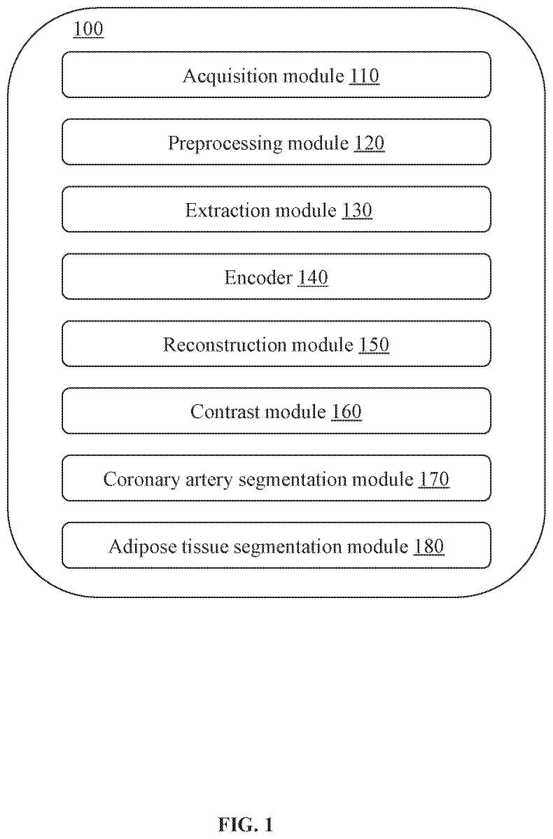

FIG. 1 is a schematic module diagram of a system for segmentation of coronary arteries and pericoronary adipose tissue based on self-supervised learning according to some embodiments of the present disclosure. As shown in FIG. 1, a system 100 for segmentation of coronary arteries and pericoronary adipose tissue based on self-supervised learning (also referred to as a segmentation system) includes an acquisition module 110, a preprocessing module 120, an extraction module 130, an encoder 140, a reconstruction module 150, a contrast module 160, a coronary artery segmentation module 170, and an adipose tissue segmentation module 180.

The acquisition module 110 is a core data input component of the segmentation system and is configured to acquire coronary computed tomography angiography (CCTA) images used for model training. The acquisition module 110 may acquire labeled CCTA images (referred to as labelled images) and unlabeled CCTA images (also referred to as unlabelled images). To ensure the generalizability of the segmentation system, the acquisition module 110 may integrate CCTA data obtained from multiple imaging devices, so that a training set of the CCTA images covers cases of different ages, sexes, and disease severities.

The preprocessing module 120 is a processing unit configured to implement data normalization and standardization, and is responsible for preprocessing the CCTA images to satisfy input requirements of a model. The preprocessing includes, but is not limited to, cropping, normalization, and denoising processing. The preprocessing module 120 can effectively reduce noise interference in the training set, unify data distribution, and accelerate a convergence speed during model training.

The extraction module 130 is a conversion unit configured to convert labels of the CCTA images into segmentation masks. The extraction module 130 may convert labels of the CCTA images (for example, regions of coronary arteries, pericoronary adipose tissue, and background) into segmentation masks suitable for model training. For example, pixels corresponding to a target region may be assigned a value of 1, while pixels corresponding to a background region may be assigned a value of 0, thereby forming a binarized mask. The obtained segmentation mask is used as a supervision signal for a subsequent coronary artery segmentation module to guide the encoder and other components to learn distribution characteristics of the coronary arteries and the pericoronary adipose tissue.

The encoder 140 is a prediction model configured to determine distributions of the coronary arteries and the pericoronary adipose tissue, and is responsible for identifying the coronary arteries and the pericoronary adipose tissue from an image to be segmented. An initial encoder needs to undergo joint pre-training and joint training, during which network parameters are iteratively optimized, so as to finally obtain the encoder 140 suitable for medical image segmentation.

The reconstruction module 150 is a parameter adjustment unit for model training. Based on an output of the encoder 140, the reconstruction module 150 dynamically updates the network parameters of the encoder 140 by using a backpropagation mechanism, such that the encoder 140 gradually learns anatomical patterns of the CCTA images (for example, morphological trajectories of coronary vessels and distribution patterns of pericoronary adipose tissue).

The contrast module 160 is a strategy evaluation unit for model training. The contrast module 160 compares outputs of the encoder 140 before and after adjustment of the network parameters, and guides the reconstruction module 150 to adjust a parameter optimization strategy, thereby improving model training speed and accuracy of the reconstruction module 150 and enhancing a capability of the encoder 140 to discriminate CCTA images.

The reconstruction module 150 and the contrast module 160 jointly operate during a pre-training process of the encoder 140. The reconstruction module 150 optimizes the network parameters of the encoder, and the contrast module 160 evaluates and guides the parameter optimization strategy of the reconstruction module 150, thereby ensuring effectiveness of parameter adjustment and jointly improving the capability of the encoder 140 to discriminate the CCTA images.

The coronary artery segmentation module 170 and the adipose tissue segmentation module 180 are working units configured to process an image to be segmented. The coronary artery segmentation module 170 is configured to output a coronary artery segmentation result of the image to be segmented, and the adipose tissue segmentation module 180 is configured to output an adipose tissue segmentation result of the image to be segmented.

The coronary artery segmentation module 170 may perform forward inference on the preprocessed image to be segmented based on the encoder, and output the coronary artery segmentation result (e.g., a pixel value of 1 indicates a coronary artery, and a pixel value of 0 indicates background).

The adipose tissue segmentation module 180 may output a pericoronary adipose tissue segmentation result based on the coronary artery segmentation result through skeletonization and threshold segmentation techniques.

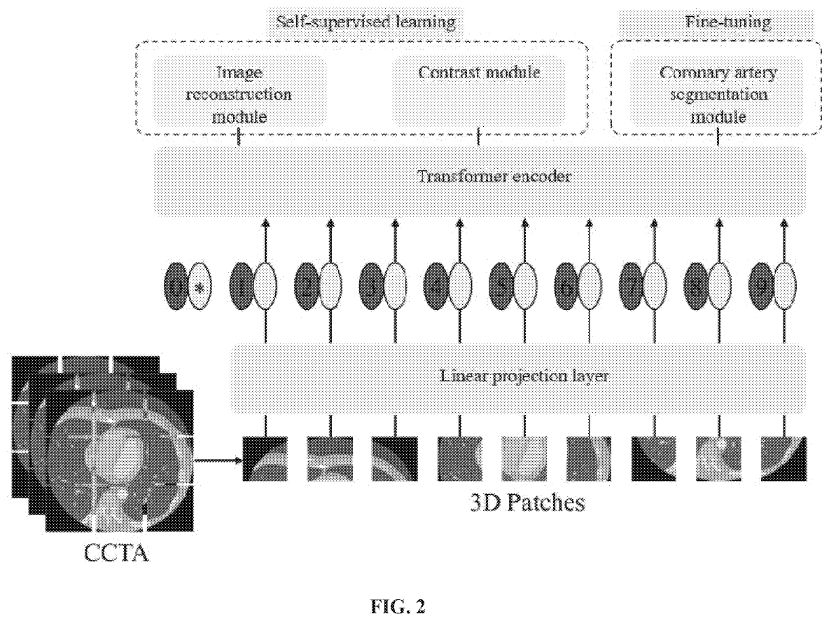

For further descriptions of the above modules, reference may be made to FIGS. 2 to 4 and the related descriptions thereof.

It should be understood that the system for segmentation of coronary arteries and pericoronary adipose tissue based on self-supervised learning and the modules thereof shown in FIG. 1 may be implemented in various manners. It should be noted that the above descriptions of the system 100 and the modules thereof are merely for convenience of description and do not limit the present disclosure to the scope of the illustrated embodiments. It can be understood that, for those skilled in the art, after understanding the principle of the segmentation system, various combinations of the modules may be made without departing from the principle, or subsystems may be formed and connected with other modules. In some embodiments, the acquisition module 110, the preprocessing module 120, the extraction module 130, the encoder 140, the reconstruction module 150, the contrast module 160, the coronary artery segmentation module 170, and the adipose tissue segmentation module 180 disclosed in FIG. 1 may be different modules in a system, or one module may implement functions of two or more of the above modules. For example, the modules may share a same storage module, or each module may have a respective storage module. Such variations are all within the scope of protection of the present disclosure.

FIG. 2 is a schematic diagram illustrating a training process of the segmentation system for coronary arteries and pericoronary adipose tissue based on self-supervised learning according to some embodiments of the present disclosure.

In some embodiments, the acquisition module is configured to acquire a training set of coronary computed tomography angiography (CCTA) images, the training set including labeled images and unlabeled images. The preprocessing module is configured to preprocess the training set and preprocess an image to be segmented. The extraction module is configured to extract segmentation masks from the preprocessed labeled images. The reconstruction module, the contrast module, and the encoder are configured to perform joint pre-training based on the preprocessed training set to obtain a pre-trained encoder. The coronary artery segmentation module and the pre-trained encoder are configured to perform joint training based on the segmentation masks to obtain a trained encoder. The encoder and the coronary artery segmentation module are configured to perform coronary artery segmentation on the preprocessed image to be segmented to obtain a coronary artery segmentation result of the image to be segmented. The adipose tissue segmentation module is configured to process the coronary artery segmentation result to obtain an adipose tissue segmentation result of the image to be segmented.

In some embodiments, the acquisition module is configured to acquire a training set of CCTA images, the training set including labeled images and unlabeled images.

A coronary computed tomography angiography (CCTA) image is a medical image obtained based on CT scanning. The CCTA image includes three-dimensional imaging data of a patient's coronary arteries and surrounding tissues, and is used to reflect anatomical structures and lesion information of the coronary arteries.

In some embodiments, the CCTA images include labeled CCTA images, i.e., labeled images, and unlabeled CCTA images, i.e., unlabeled images. Labels of the CCTA images include regional annotations of coronary arteries, pericoronary adipose tissue, and background. The acquisition module may acquire a large number of CCTA images from CT devices, select a portion of the CCTA images and send them to medical personnel, and the medical personnel annotate regions such as coronary arteries and pericoronary adipose tissue in the CCTA images to obtain a portion of labeled images, while the remaining unannotated CCTA images are used as the unlabeled images.

In some embodiments, the preprocessing module is configured to preprocess labeled images and unlabeled images in the training set, and to preprocess an image to be segmented.

The image to be segmented is a clinical CCTA image for which a segmentation result is to be obtained. The acquisition module may acquire, from a CT device, the image to be segmented of a current patient, and transmit the image to the preprocessing module for preprocessing.

Preprocessing refers to operations for normalization and standardization of the CCTA images. In some embodiments, specific steps of the preprocessing may be determined based on actual requirements. The preprocessing includes, but is not limited to, white balance adjustment, normalization, and denoising processing.

In some embodiments, the preprocessing module is further configured to crop and standardize the labeled images and the unlabeled images in the training set, so as to unify the labeled images and the unlabeled images to a same size.

In some embodiments, the preprocessing further includes cropping and standardization. Cropping is used to remove irrelevant regions in the CCTA images, and standardization is a processing step for converting CCTA images from different sources into images having a same size.

In some embodiments, a target size of the CCTA images may be determined based on actual requirements.

In some embodiments, the preprocessing module may convert the CCTA images into CCTA images having a size of 512×512×256 pixels. The preprocessing module may alternatively convert the CCTA images into other sizes different from 512×512×256 pixels.

In some embodiments, by cropping and standardizing the labeled images and the unlabeled images in the training set so as to unify the labeled images and the unlabeled images to a same size, irrelevant regions in the CCTA images may be removed through cropping so as to reduce noise interference. Further, through standardization, CCTA images acquired from different devices may be unified to a same size, thereby eliminating differences caused by different data sources and improving data consistency.

In some embodiments, the extraction module is configured to extract segmentation masks from preprocessed labeled images.

A segmentation mask refers to a binarized image for marking a coronary artery region. Because labels of CCTA images may include various styles such as region annotations and boundary annotations, which are not conducive to interpretation and training by the encoder, the extraction module may convert the labels into segmentation masks that are easier for the model to interpret, so as to guide training of the encoder.

In a coronary artery segmentation task, the extraction module may generate a segmentation mask having the same size as the CCTA image. Pixels of the segmentation mask correspond one-to-one with pixels of the labeled CCTA image. When a pixel in the labeled CCTA image belongs to a coronary artery, the corresponding pixel in the segmentation mask is marked as 1; otherwise, the corresponding pixel is marked as 0.

In some embodiments, for any labeled CCTA image, the extraction module may denote a segmentation mask corresponding to the labeled CCTA image as:

$${y}_{g t} ∈ {R}^{H \times W \times C} ,$$

where H and W denote a pixel height and a pixel width of the segmentation mask, respectively, and C denotes a count of foreground classes to be segmented in the CCTA image. In the coronary artery segmentation task, the count of foreground classes C is 1. For each pixel in the CCTA image, if the pixel belongs to a coronary artery, the corresponding pixel in the segmentation mask is marked as 1; otherwise, the pixel is marked as 0.

In some embodiments, the reconstruction module, the contrast module, and the encoder are configured to perform joint pre-training based on the preprocessed training set so as to obtain a pre-trained encoder.

The encoder is a prediction model configured to determine distributions of coronary arteries and pericoronary adipose tissue. In some embodiments, the encoder may be a machine learning model. For example, the encoder may include any one of a Transformer model, other customized model architectures, or any combinations thereof.

In some embodiments, a model architecture of the encoder may be determined based on practical requirements. For example, the encoder may include one or more encoding units.

In some embodiments, the encoder includes twelve encoding units connected in series, and the encoder is further configured to: uniformly partition each of the preprocessed labeled images and each of the preprocessed unlabeled images into a plurality of non-overlapping patches, respectively, wherein each of the patches has a size of P×P×P, wherein P is a preset resolution; flatten the partitioned patches and pass the flattened patches through a linear projection layer, then pass an output of the linear projection layer through a positional embedding layer, and perform positional embedding on the output of the linear projection layer based on the positional embedding layer to obtain positional embedding results corresponding to the patches; and input the positional embedding results of the patches into the encoder, wherein within the encoder, the positional embedding results sequentially pass through the twelve encoding units connected in series, and feature maps output by a third encoding unit, a sixth encoding unit, a ninth encoding unit, and a twelfth encoding unit are stored to obtain a set of feature maps.

An encoding unit is a submodule of the encoder. A module structure of the encoding unit may be determined based on practical requirements.

In some embodiments, in order to improve an operating speed of the model, the encoder may uniformly divide a large-sized CCTA image into a plurality of non-overlapping patches, each patch having a size of P×P×P, where P is a preset patch resolution.

Flattening a patch refers to an operation of converting a three-dimensional patch (P×P×P) into a one-dimensional vector. Patch flattening enables the patch to conform to an input format of the linear projection layer.

The linear projection layer performs an operation of projecting the one-dimensional vector into an embedding space. The linear projection layer may convert original pixel information contained in the one-dimensional vector into a low-dimensional sequence that can be interpreted by the model.

In some embodiments, the encoder may flatten each of the divided patches and, through the linear projection layer, project the flattened results into an embedding space so as to create a sequence X∈R^{N×d}, where N denotes a count of patches and d denotes an embedding dimension.

In some embodiments, an output of the linear projection layer is further passed through a positional embedding layer, which performs positional embedding on the output of the linear projection layer to obtain positional embedding results corresponding to the patches.

The positional embedding layer is a marking layer configured to indicate relative positions among the patches. In some embodiments, because the large-sized CCTA image is uniformly divided into multiple non-overlapping patches, relative positional information among the patches may be lost during the division process. Therefore, the output of the linear projection layer is processed by the positional embedding layer, that is, positional embedding is performed on the output of the linear projection layer to obtain the positional embedding result corresponding to each patch. For example, during positional embedding, the positional embedding layer may additionally add position coordinates of each patch in the original CCTA image to the sequence X∈R^{N×d}, thereby obtaining positional embedding results including the sequence X∈R^{N×d} and the position coordinates.

In some embodiments, the encoder uses positional embedding results of all patches as inputs of the encoder. Within the encoder, the positional embedding results of all patches sequentially pass through the twelve encoding units connected in series, and feature maps output from the third encoding unit, the sixth encoding unit, the ninth encoding unit, and the twelfth encoding unit are stored, thereby obtaining, for each patch, a set of feature maps.

A feature map is a set of features output from the third encoding unit, the sixth encoding unit, the ninth encoding unit, and the twelfth encoding unit after the positional embedding results are processed by the twelve encoding units. Through multiple layers of abstraction and transformation, the feature maps retain key information such as spatial positions and image semantics contained in the positional embedding results, and serve as inputs to the image reconstruction module in subsequent steps.

In some embodiments, the encoder includes twelve encoding units connected in series, and the image is divided into non-overlapping patches with positional embedding introduced. By decomposing a large-sized CCTA image into small-sized patches, a processing speed of large-scale three-dimensional medical images can be improved. Further, by storing the feature maps output from the third encoding unit, the sixth encoding unit, the ninth encoding unit, and the twelfth encoding unit, semantic information ranging from low-level to high-level (such as edges, textures, and anatomical structures) is preserved, thereby providing rich feature support for subsequent coronary artery segmentation and pericoronary adipose tissue segmentation.

In some embodiments, each encoding unit of the encoder includes a first normalization layer, a multi-head attention layer, a second normalization layer, and a multi-layer perceptron.

A normalization layer is a layer configured to perform normalization processing on an input feature of the encoding unit, so as to compress a value range of the feature data into a specific interval (for example, an interval of [0,1]). The normalization layer includes, but is not limited to, a Batch Normalization (BN) layer and a Layer Normalization (LN) layer.

The multi-head attention layer calculates correlation weights among different positions of the input feature through multiple parallel attention heads.

The multi-layer perceptron is a perception layer composed of fully connected layers and nonlinear activation functions (such as ReLU). The multi-layer perceptron may perform nonlinear transformation and dimensional mapping on the normalized features, thereby extracting more abstract high-level semantic features.

In some embodiments, the encoder is further configured to: for any encoding unit among the encoding units of the encoder, input an input feature of the encoding unit into the first normalization layer, and take an output of the first normalization layer as an input of the multi-head attention layer; add an output of the multi-head attention layer and the input feature of the encoding unit to obtain a first sum result; input the first sum result into the second normalization layer, and take an output of the second normalization layer as an input of the multilayer perceptron; and add an output of the multilayer perceptron and the first sum result to obtain a second sum result, and take the second sum result as an output of the encoding unit.

In some embodiments, for the twelve encoding units connected in series, an output of the first encoding unit is used as an input to the second encoding unit. The working process within the second encoding unit is the same as that within the first encoding unit. An output of the second encoding unit is then used as an input to the third encoding unit, and so on, such that the working processes of the twelve encoding units are identical. The encoder may store the feature maps output by the third encoding unit, the sixth encoding unit, the ninth encoding unit, and the twelfth encoding unit, thereby obtaining a set of feature maps for each patch.

In some embodiments, the encoder may further store the feature map output by each encoding unit, thereby obtaining a set of feature maps corresponding to each patch.

In some embodiments, by configuring each encoding unit to include the first normalization layer, the multi-head attention layer, the second normalization layer, and the multilayer perceptron, the problem of gradient vanishing during network training can be effectively alleviated, thereby promoting deep propagation of feature information. The multi-head attention mechanism enhances the model's ability to capture both local and global correlations in images (e.g., spatial relationships between coronary artery branches and surrounding tissues), while the multilayer perceptron improves the feature abstraction capability through nonlinear transformations. The synergistic effect of the multi-head attention mechanism and the multilayer perceptron ensures that the encoder can learn more discriminative medical image features, thereby improving segmentation accuracy.

In some embodiments, the reconstruction module, the comparison module, and the encoder are configured to perform joint pre-training based on the preprocessed labeled images and the preprocessed unlabeled images, so as to obtain a pre-trained encoder.

The joint pre-training refers to a process of training the initial encoder to obtain the pre-trained encoder. The joint pre-training belongs to self-supervised learning and can utilize a large number of unlabeled images for training, thereby effectively reducing the cost of data annotation.

In some embodiments, the joint pre-training of the encoder, the reconstruction module, and the contrastive module includes:
S41: initializing a pre-training epoch t to 1.
S42: flattening patches corresponding to the preprocessed training set, and sequentially processing the flattened patches through the linear projection layer, the positional embedding layer, and the encoder to obtain a set of feature maps corresponding to each patch (i.e., each patch corresponds to a respective set of feature maps).

For more details regarding the flattening operation, the linear projection layer, the positional embedding layer, and the encoder, reference may be made to the foregoing description related to the encoding unit.

S43: for an i-th patch, which is denoted as zᵢ, obtaining a reconstruction result \hat{z}ᵢ of the i-th patch zᵢ output from the reconstruction module by inputting the i-th patch zᵢ and the corresponding set of feature maps into the reconstruction module.

The reconstruction result \hat{z}ᵢ is a low-dimensional vector predicted by the reconstruction module, and includes predicted features of input data generated by the reconstruction module.

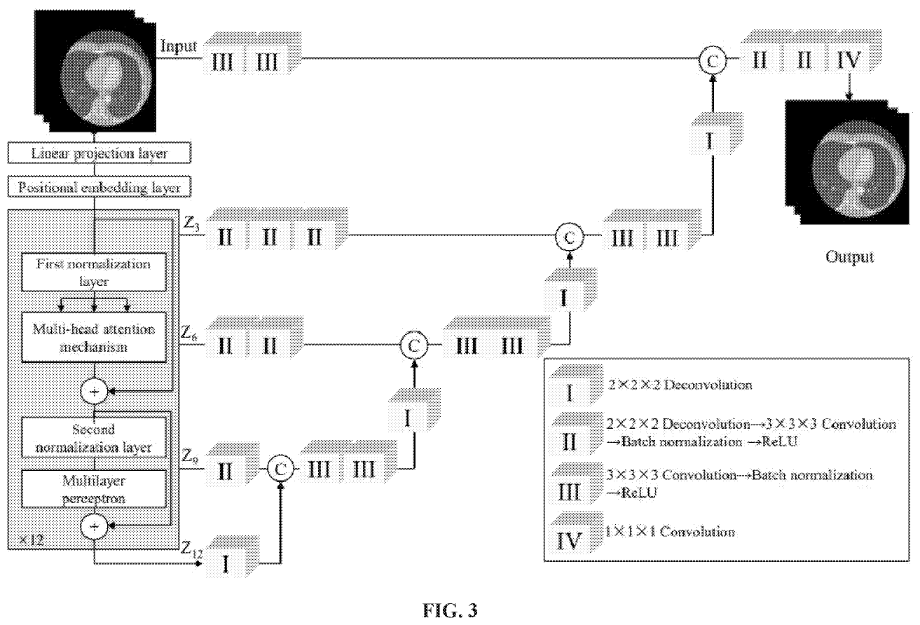

FIG. 3 is a schematic structural diagram of an encoder and a reconstruction module according to some embodiments of the present disclosure. In some embodiments, obtaining the reconstruction result \hat{z}ᵢ of the i-th patch zᵢ output by the reconstruction module includes:
S43-1: using the i-th patch zᵢ as an input to a first convolutional layer, and sequentially processing the input through the first convolutional layer, a first BN layer, a first ReLU layer, a second convolutional layer, a second BN layer, and a second ReLU layer to obtain an output of the second ReLU layer, the output being denoted as a.

That is to say, the i-th patch zᵢ is first processed by the first convolutional layer, an output of the first convolutional layer is used as an input to the first BN layer, an output of the first BN layer is used as an input to the first ReLU layer, an output of the first ReLU layer is used as an input to the second convolutional layer, an output of the second convolutional layer is used as an input to the second BN layer, and an output of the second BN layer is used as an input to the second ReLU layer, where the output of the second ReLU layer is denoted as a.

S43-2: sequentially processing a feature map output by the third encoding unit through a first deconvolution sub-unit, a second deconvolution sub-unit, and a third deconvolution sub-unit to obtain an output b of the third deconvolution sub-unit.

That is to say, the feature map output by the third encoding unit is first processed by the first deconvolution sub-unit, an output of the first deconvolution sub-unit is used as an input to the second deconvolution sub-unit, an output of the second deconvolution sub-unit is used as an input to the third deconvolution sub-unit, and the output of the third deconvolution sub-unit is denoted as b.

In some embodiments, a deconvolution sub-unit includes a deconvolution layer, a convolution layer, a BN layer, and a ReLU layer sequentially connected in series.

The convolution layer is a network layer configured to perform sliding-window computation on input features by using a convolution kernel having a preset size, multiplying pixel values of a local region with weights of the convolution kernel and accumulating the products to generate output features. The convolution layer can significantly reduce a number of network parameters and lower computational complexity.

The deconvolution layer is a network layer configured to restore a resolution or expand dimensions of input features by performing upsampling on the input features and applying a convolution operation.

The Batch Normalization (BN) layer is a normalization layer configured to perform normalization processing on input features so as to adjust feature data to a unified distribution (for example, within a range of [0,1]).

The ReLU layer is a nonlinear activation function represented as f(x)=max(0,x). The ReLU layer sets negative input values to zero while keeping non-negative input values unchanged. The ReLU layer enables a network to fit complex nonlinear relationships.

In some embodiments, kernel sizes of the deconvolution layer and the convolution layer in the first deconvolution sub-unit may be preset based on prior experience.

In some embodiments, a deconvolution kernel size of the deconvolution layer in the first deconvolution sub-unit is 2×2×2, and a convolution kernel size of the convolution layer in the first deconvolution sub-unit is 3×3×3.

In some embodiments, structures and working processes of the second deconvolution sub-unit, the third deconvolution sub-unit, a fourth deconvolution sub-unit, a fifth deconvolution sub-unit, and a sixth deconvolution sub-unit are the same as those of the first deconvolution sub-unit.

In some embodiments, by configuring the deconvolution sub-unit to include a deconvolution layer, a convolution layer, a BN layer, and a ReLU layer sequentially connected in series, the convolution layer refines feature details, the BN layer accelerates training convergence and prevents overfitting, and the ReLU layer introduces nonlinear transformations to enhance feature representation capability. This structure helps reduce information loss during an upsampling process, improves spatial resolution and a signal-to-noise ratio of feature maps, and provides higher-quality feature inputs for subsequent reconstruction and segmentation tasks.

S43-3, processing the feature map output from the sixth encoding unit sequentially through the fourth deconvolution sub-unit and the fifth deconvolution sub-unit to obtain an output c of the fifth deconvolution sub-unit.

Specifically, the feature map output from the sixth encoding unit is first processed by the fourth deconvolution sub-unit, and an output of the fourth deconvolution sub-unit is used as an input of the fifth deconvolution sub-unit; an output of the fifth deconvolution sub-unit is denoted as c.

S43-4, processing a feature map output from the ninth encoding unit through a sixth deconvolution sub-unit to obtain an output d of the sixth deconvolution sub-unit.

S43-5, processing a feature map output from the twelfth encoding unit through a first upsampling unit (i.e., a deconvolution layer) to obtain an output e of the first upsampling unit.

S43-6, concatenating the output e and the output d, and sequentially processing a result of the concatenation through a third convolution layer, a third BN layer, a third ReLU layer, a fourth convolution layer, a fourth BN layer, a fourth ReLU layer, and a second upsampling unit to obtain an output i′ of the second upsampling unit.

Specifically, the result of the concatenation in step S43-6 is first processed by the third convolution layer, and an output of the third convolution layer is used as an input of the third BN layer; an output of the third BN layer is used as an input of the third ReLU layer; an output of the third ReLU layer is used as an input of the fourth convolution layer; an output of the fourth convolution layer is used as an input of the fourth BN layer; an output of the fourth BN layer is used as an input of the fourth ReLU layer; and an output of the fourth ReLU layer is then processed by the second upsampling unit, with an output of the second upsampling unit being denoted as i′.

S43-7, concatenating the output c and the output i′, and sequentially processing a result of the concatenation through a fifth convolution layer, a fifth BN layer, a fifth ReLU layer, a sixth convolution layer, a sixth BN layer, a sixth ReLU layer, and a third upsampling unit to obtain an output j′ of the third upsampling unit.

Specifically, the result of the concatenation in step S43-7 is first processed by the fifth convolution layer, and an output of the fifth convolution layer is used as an input of the fifth BN layer; an output of the fifth BN layer is used as an input of the fifth ReLU layer; an output of the fifth ReLU layer is used as an input of the sixth convolution layer; an output of the sixth convolution layer is used as an input of the sixth BN layer; an output of the sixth BN layer is used as an input of the sixth ReLU layer; and an output of the sixth ReLU layer is then processed by the third upsampling unit, with the output of the third upsampling unit being denoted as j′.

S43-8, concatenating the output b and the output j′, and sequentially processing a result of the concatenation through a seventh convolution layer, a seventh BN layer, a seventh ReLU layer, an eighth convolution layer, an eighth BN layer, an eighth ReLU layer, and a fourth upsampling unit to obtain an output k of the fourth upsampling unit.

Specifically, the result of the concatenation in step S43-8 is first processed by the seventh convolution layer, and an output of the seventh convolution layer is used as an input of the seventh BN layer; an output of the seventh BN layer is used as an input of the seventh ReLU layer; an output of the seventh ReLU layer is used as an input of the eighth convolution layer; an output of the eighth convolution layer is used as an input of the eighth BN layer; an output of the eighth BN layer is used as an input of the eighth ReLU layer; and an output of the eighth ReLU layer is then processed by the fourth upsampling unit, with the output of the fourth upsampling unit being denoted as k.

S43-9, concatenating the output k and the output a, and sequentially processing a result of the concatenation through a ninth convolution layer, a ninth BN layer, a ninth ReLU layer, a tenth convolution layer, a tenth BN layer, a tenth ReLU layer, and an eleventh convolution layer to finally obtain the reconstruction result \hat{z}ᵢ of the i-th patch zᵢ.

Specifically, the result of the concatenation in step S43-9 is first processed by the ninth convolution layer, and an output of the ninth convolution layer is used as an input of the ninth BN layer; an output of the ninth BN layer is used as an input of the ninth ReLU layer; an output of the ninth ReLU layer is used as an input of the tenth convolution layer; an output of the tenth convolution layer is used as an input of the tenth BN layer; and an output of the tenth BN layer is used as an input of the tenth ReLU layer. An output of the tenth ReLU layer is then used as an input of the eleventh convolution layer, and an output of the eleventh convolution layer is used as the reconstruction result \hat{z}ᵢ of the i-th patch zᵢ.

In some embodiments, the reconstruction module adopts a multi-scale feature fusion strategy and a progressive upsampling strategy, which facilitates accurate restoration of the spatial structure and anatomical details of the input patch, and particularly improves the morphological reconstruction of fine coronary branches. By alternately employing the deconvolution sub-units and the convolution layers, artifacts generated during the upsampling process can be effectively suppressed, thereby improving the clarity of the reconstruction result.

S44, inputting the set of feature maps corresponding to the i-th patch zᵢ into a contrast module, and sequentially processing the set of feature maps through a global average pooling layer and a linear projection layer of the contrast module to obtain a feature embedding zᵢ′ of the i-th patch zᵢ.

The feature embedding zᵢ′ is a low-dimensional vector obtained by processing the set of feature maps by the contrast module, and represents a true feature extracted by the model.

In some embodiments, the feature embedding zᵢ′ of the i-th patch zᵢ is expressed as:

$${z}_{i}^{′} = h ( g ( f ) ) ,$$

where f denotes the set of feature maps corresponding to the i-th patch zᵢ, g(f) denotes a feature vector output by the global average pooling layer, h denotes the linear projection layer, and zᵢ′ denotes the feature embedding output by the linear projection layer.

In some embodiments, by setting the feature embedding zᵢ′ as zᵢ′=h(g(f)), the global average pooling layer can be used to convert multi-scale feature maps into a compact feature vector, which is then mapped to a low-dimensional embedding through the linear projection layer. This configuration effectively reduces the feature dimensionality while preserving key semantic information. This feature embedding scheme enhances the capability of the contrast module to capture global image features, enabling the encoder to learn more discriminative cross-image feature associations and improving the generalization performance of the model.

S45, determining a total loss function L based on the reconstruction result \hat{z}ᵢ and the feature embedding zᵢ, performing backpropagation based on the total loss function L, and adjusting network parameters in the initial encoder, the reconstruction module, and the contrast module; in response to convergence of the total loss function L, pausing the backpropagation.

Backpropagation is an algorithm that computes gradients of parameters from an output layer to an input layer based on a loss function, so as to update the network parameters.

In some embodiments, the encoder, the reconstruction module, and the contrast module perform layer-by-layer backward computation of gradients of parameters (such as weights and biases) of the initial encoder starting from the output layer based on the total loss function L, where the gradients reflect a magnitude and a direction of an influence of parameter variation on the loss function. The encoder, the reconstruction module, and the contrast module determine an adjustment magnitude and direction of the parameters of the initial encoder according to the magnitude and sign of the gradients. For example, when a gradient is positive, the parameter is decreased, and when the gradient is negative, the parameter is increased. A larger absolute value of the gradient corresponds to a larger adjustment magnitude of the parameter, and vice versa.

It is known that the total loss function L has two possible states: non-convergence and convergence. In some embodiments, in response to the total loss function L not converging, the encoder, the reconstruction module, and the contrast module repeatedly perform gradient computation and parameter adjustment processes, such that the network parameters are gradually optimized in a direction that reduces the total loss function L. In response to the total loss function L converging, the backpropagation is paused.

The total loss function L is a function that measures a difference between a predicted value of the initial encoder and a ground truth value, and is a key indicator for evaluating model performance in machine learning and deep learning.

In some embodiments, the closer the reconstruction result \hat{z}ᵢ of zᵢ is to zᵢ, the smaller the total loss function L, indicating that the encoder has more sufficiently learned features in the CCTA image such as coronary vascular morphology, lumen boundaries, and pericoronary adipose tissue density. The more the reconstruction result \hat{z}ᵢ of zᵢ deviates from zᵢ, the larger the total loss function L, indicating that the model has insufficient accuracy in anatomical structure reconstruction or that confusion exists in feature learning.

In some embodiments, the total loss function L is defined as:

$$L = {L}_{recon} + {L}_{c o n t r a s t} ,$$

where L_{recon} denotes a reconstruction loss and L_{contrast} denotes a patch contrast loss.

The reconstruction loss L_{recon} is a loss function obtained based on the reconstruction result \hat{z}ᵢ of a single zᵢ. The contrast loss L_{contrast} is a loss function obtained based on feature embeddings of a plurality of patches in the same CCTA image.

$${L}_{recon} = {\sum }_{{z}_{i} ∈ M} ‖ {z}_{i} - \text{?} {‖}_{2}^{2} ,$$

where M is a set of all patches, and I·I₂ denotes an L2 norm.

$${L}_{c ontrast} = {\sum }_{{z}_{i} ∈ M} - log \frac{exp ( s im ( {z}_{i}^{′} , {z}^{+} )}{{\sum }_{{z}_{j} ∈ N ( i )} exp ( s im ( ( {z}_{i}^{′} , ( {z}_{j}^{′} ) )} ,$$

where zᵢ′ is the feature embedding of zᵢ, z⁺ is a feature embedding of another patch from a same CCTA image as zᵢ, sim (zᵢ′, z⁺) represents a similarity between zᵢ′ and z⁺, N(i) represents a set of all patches other than zᵢ, sim (zᵢ′, zⱼ′) represents a similarity between zᵢ′ and zⱼ′, and zⱼ′ is a feature embedding of a j-th patch different from zᵢ.

In some embodiments, by setting the total loss function L as L=L_{recon}+L_{contrast}, the reconstruction loss L_{recon} mainly focuses on low-level visual features of a single patch (for example, edges, textures, and anatomical details), while the contrast loss L_{contrast} mainly focuses on high-level semantic features among multiple patches (for example, tissue distribution, spatial relationships, and pathological attributes). Through a synergistic effect of the two losses, the encoder is optimized from two dimensions, namely structural fidelity and semantic discrimination, thereby ultimately enabling efficient segmentation of coronary arteries and pericoronary adipose tissue.

S46: determining whether the pre-training epoch t has reached a maximum pre-training epoch. In response to determining that the pre-training epoch t has not reached the maximum pre-training epoch, re-partitioning the preprocessed training set into patches, updating the pre-training epoch t to a pre-training epoch t+1, and continuing the joint pre-training for the pre-training epoch t+1. In response to determining that the pre-training epoch t has reached the maximum pre-training epoch, completing the joint pre-training to obtain the pre-trained encoder.

In some embodiments, it is known that two situations exist: the pre-training epoch t reaches the maximum pre-training epoch, or the pre-training epoch t does not reach the maximum pre-training epoch.

In some embodiments, in response to the pre-training epoch t not reaching the maximum pre-training epoch, the joint pre-training is not yet completed. The reconstruction module, the contrast module, and the encoder may re-partition the preprocessed training set into patches, and the obtained patches are used for joint pre-training in the pre-training epoch t+1.

In some embodiments, when performing patch partitioning in the pre-training epoch t+1, labeled images and unlabeled images different from those used in the pre-training epoch t may be selected from the preprocessed training set for partitioning. Alternatively, the same labeled images and unlabeled images as those used in the pre-training epoch t may be selected from the preprocessed training set, but partitioning may be performed using partitioning parameters (e.g., different patch sizes) different from those in the pre-training epoch t, thereby obtaining a plurality of patches different from those in the pre-training epoch t, which serve as training samples and labels for the initial encoder.

In some embodiments, in response to the pre-training epoch t reaching the maximum pre-training epoch, the joint pre-training is completed, and the pre-trained encoder is obtained. The pre-trained encoder may be further converted into a trained encoder through subsequent joint training.

In some embodiments, the coronary artery segmentation module and the pre-trained encoder are configured to perform joint training based on segmentation masks, so as to obtain the trained encoder.

The joint training refers to a process of performing secondary training on the pre-trained encoder to obtain the encoder. The joint training belongs to supervised learning, and network parameters of the pre-trained encoder may be fine-tuned based on professional annotations of CCTA images, thereby ensuring professionalism and accuracy of the encoder.

In some embodiments, the coronary artery segmentation module may train and test the pre-trained encoder based on the segmentation mask to obtain the trained encoder. For example, the coronary artery segmentation module may use zᵢ as a training sample and the segmentation mask as a label, input the training sample into the pre-trained encoder to obtain a set of feature maps of zᵢ output by the pre-trained encoder, input zᵢ and the set of feature maps corresponding to zᵢ into the coronary artery segmentation module, construct a loss function based on a coronary artery segmentation result output by the coronary artery segmentation module and the label, and iteratively update parameters of the pre-trained encoder based on the loss function until a preset condition is satisfied, at which point training ends and a trained encoder is obtained. The preset condition may include, but is not limited to, convergence of the loss function, a training epoch reaching a threshold, or the like.

In some embodiments, the joint training of the coronary artery segmentation module and the pre-trained encoder includes:
S51: initializing a training epoch p to 1.
S52: flattening patches corresponding to preprocessed labeled images, and sequentially processing the flattened patches through the linear projection layer, the position embedding layer, and the encoder to obtain a set of feature maps corresponding to each of the patches.

The manner of obtaining the set of feature maps of the patch in S52 is similar to the manner of obtaining the set of feature maps of the patch in S42, and reference may be made to the related description of S42 above.

S53: for the i-th patch, denoting the patch as zᵢ, and inputting zᵢ and the set of feature maps corresponding to zᵢ into the coronary artery segmentation module.

The coronary artery segmentation module is a prediction model configured to determine a coronary artery segmentation result. The structure of the coronary artery segmentation module may be determined according to practical requirements.

In some embodiments, the structure of the coronary artery segmentation module is substantially the same as that of the reconstruction module. For the portion preceding the tenth ReLU layer, the structure of the coronary artery segmentation module is identical to that of the reconstruction module. The difference lies in that, in the reconstruction module, an output of the tenth ReLU layer is processed by an eleventh convolutional layer to finally obtain the reconstruction result 21 of [symbol] . In the coronary artery segmentation module, the output of the tenth ReLU layer is sequentially processed by an upsampling unit, a ReLU layer, and a softmax activation function layer, and the coronary artery segmentation result is output by the softmax activation function layer.

The softmax activation function layer is a function transformation layer configured to convert a numerical vector into a probability distribution vector, so as to perform binary classification of pixel values and distinguish a coronary artery region from a background region. For example, for pixels at an edge of a coronary artery lumen, the softmax function may output a probability close to 1, while for pixels in the background region, the softmax function may output a probability close to 0, thereby enabling accurate discrimination between coronary arteries and background tissues (such as myocardium and vascular adventitia).

S54: determining a segmentation loss L′ based on the coronary artery segmentation results of the patches predicted by the coronary artery segmentation module and the segmentation masks corresponding to the patches, performing backpropagation based on the segmentation loss L′, adjusting network parameters in the pre-trained encoder and the coronary artery segmentation module, and pausing the backpropagation in response to convergence of the segmentation loss L′.

The manner of performing backpropagation based on the segmentation loss L′ in S54 is similar to the manner of performing backpropagation based on the total loss function L in S45, and reference may be made to the related description above.

It is known that the segmentation loss L′ may have two states: non-convergence and convergence. In some embodiments, in response to the segmentation loss L′ not converging, the coronary artery segmentation module and the pre-trained encoder repeatedly perform gradient calculation and parameter adjustment so that the network parameters are gradually optimized in a direction that reduces the segmentation loss L′. In response to the segmentation loss L′ converging, the backpropagation is paused.

The segmentation loss L′ is a function used to measure a difference between a predicted value of the coronary artery segmentation module and a ground truth value, and is a key indicator for evaluating model performance in machine learning and deep learning.

In some embodiments, the closer the coronary artery segmentation result is to the segmentation mask, the smaller the segmentation loss L′, indicating that the pre-trained encoder has more sufficiently learned features in the CCTA image, such as coronary vessel morphology, lumen boundaries, and density of pericoronary adipose tissue. Conversely, the more the coronary artery segmentation result deviates from the segmentation mask, the larger the segmentation loss L′, indicating insufficient anatomical reconstruction accuracy or confusion in feature learning by the model.

In some embodiments, the coronary artery segmentation module uses the segmentation mask as a supervisory signal to perform joint training with the pre-trained encoder. With a relatively small number of labeled images, parameters of the pre-trained encoder may be selectively fine-tuned, thereby significantly improving the accuracy and stability of the segmentation result.

In some embodiments, the segmentation loss L′ is defined as:

$${L}^{′} = {L}_{C E} + {L}_{D i c e} ,$$

where L_{CE} denotes a cross-entropy loss, and L_{Dice} denotes a Dice loss.

The cross-entropy loss is a loss function for measuring a difference between a predicted probability distribution and a ground-truth label distribution.

The Dice loss is a loss function used for evaluating an overlap degree in a segmentation task.

$${L}_{C E} = - {\sum }_{i = 1}^{N} {\sum }_{k = 1}^{K} {y}_{g {t}_{i , k}} log ( {y}_{p r e {d}_{i , k}} ) + ( 1 - {y}_{g {t}_{i , k}} ) log ( 1 - {y}_{p r e {d}_{i , k}} ) ,$$

$${L}_{D i c e} = 1 - \frac{2 {\sum }_{i = 1}^{N} {\sum }_{k = 1}^{K} {y}_{g {t}_{i , k}} {y}_{p r e {d}_{i , k}}}{{\sum }_{i = 1}^{N} {\sum }_{k = 1}^{K} {y}_{g {t}_{i , k}} + {\sum }_{i = 1}^{N} {\sum }_{k = 1}^{K} {y}_{p r e {d}_{i , k}}} ,$$

where N denotes a set of all patches corresponding to the labeled CCTA images, K denotes a count of pixels in a single patch, y_{gt}_{_{i,k}} denotes a ground-truth label for a k-th pixel from a segmentation mask corresponding to the i-th patch zᵢ, and y_{pred}_{_{i,k}} denotes a predicted result for the k-th pixel in the i-th patch zᵢ.

In some embodiments, the segmentation loss L′ is set as L′=L_{CE}+L_{Dice}. The cross-entropy loss L_{CE} can accurately calibrate a classification probability of coronary lumen edge pixels by minimizing a difference between the predicted probability distribution and the ground-truth label distribution, thereby ensuring that the encoder can accurately capture differences in Hounsfield Unit (HU) values between coronary arteries and background tissues (such as myocardium), and avoiding segmentation deviation caused by blurred edges. The Dice loss L_{Dice} can address a problem of pixel sparsity in fine coronary branches, forcing the encoder to learn a branch topology structure to avoid breakage or omission, thereby providing reliable regional boundaries for clinical evaluation. By combining the cross-entropy loss and the Dice loss, the pre-trained encoder can converge more effectively during a training process and segmentation accuracy can be improved.

S55, determining whether the training epoch p reaches a maximum training epoch; in response to determining that the training epoch p has not reach the maximum training epoch, re-partitioning the preprocessed training set into patches, updating the training epoch p to a training epoch p+1, and continuing the joint training for the training epoch p+1; and in response to determining that the training epoch p has reached the maximum training epoch, completing the joint training to obtain the trained encoder.

In some embodiments, it is known that the training epoch p may correspond to two cases: the training epoch p has not reached the maximum training epoch and the training epoch p has reached the maximum training epoch. In response to determining that the training epoch p has not reached the maximum training epoch, the joint training is not yet completed, and the coronary segmentation module and the pre-trained encoder may re-perform patch partitioning on the preprocessed labeled images, and patches obtained through the partitioning are used for the joint pre-training for the training epoch p+1.

In some embodiments, when performing patch partitioning for the training epoch p+1, labeled images different from those used in the training epoch p may be selected from the preprocessed labeled images for partitioning. Alternatively, the same labeled images as those used in the training epoch p may be selected from the preprocessed labeled images, but partitioning may be performed using parameters different from those used in the p-th training epoch (for example, different patch sizes), thereby obtaining a plurality of patches different from those in the training epoch p, which are used as training samples and labels for the pre-trained encoder.

In some embodiments, in response to the training epoch p reaching the maximum number of training epochs, the joint training is determined to be completed, and the trained encoder is obtained.

In some embodiments, through the joint training of the coronary artery segmentation module and the pre-trained encoder, the parameters of the pre-trained encoder are fine-tuned using supervision signals provided by the segmentation masks, thereby fully utilizing the general features learned during the self-supervised learning stage and accelerating the convergence speed of the segmentation model.

In some embodiments, the encoder and the coronary artery segmentation module are configured to perform coronary artery segmentation on a preprocessed image to be segmented, thereby obtaining a coronary artery segmentation result of the image to be segmented.

In some embodiments, the preprocessing module may preprocess the image to be segmented. The steps for preprocessing the image to be segmented by the preprocessing module are similar to those for preprocessing the training set, and reference may be made to the foregoing description for details.

In some embodiments, the adipose tissue segmentation module is configured to process the coronary artery segmentation result to obtain an adipose tissue segmentation result of the image to be segmented.

The adipose tissue segmentation result is a segmentation result of pericoronary adipose tissue.

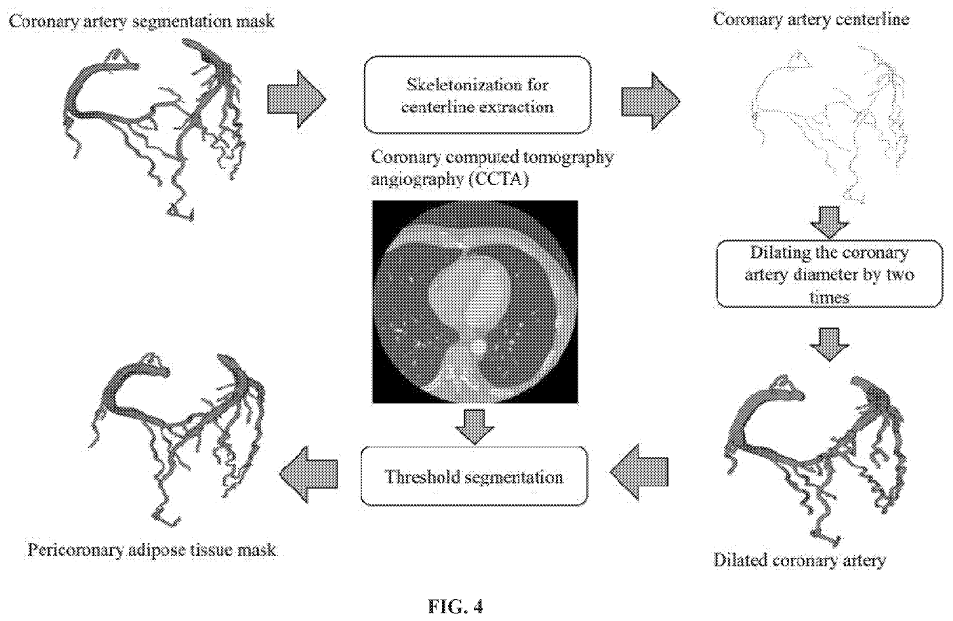

FIG. 4 is a flowchart illustrating an algorithm for adipose tissue segmentation according to some embodiments of the present disclosure. As shown in FIG. 4, the adipose tissue segmentation result is presented in the form of three-dimensional modeling, in which a dark region having a pixel value of 1 corresponds to pericoronary adipose tissue, and a light region having a pixel value of 0 corresponds to the background. The adipose tissue segmentation result can represent spatial distribution characteristics of the pericoronary adipose tissue.

The segmentation system provided in the present disclosure significantly reduces dependence on large-scale annotated data by combining self-supervised learning using unlabeled data and supervised learning using labeled data. By using a small batch of labeled images, an encoder having good segmentation performance can be trained, thereby reducing the cost of training the encoder. In addition, the segmentation system integrates the coronary artery segmentation module and the adipose tissue segmentation module, enabling automatic segmentation of coronary artery structures and pericoronary adipose tissue from CCTA images, thereby improving segmentation efficiency in clinical applications.

In some embodiments, obtaining the adipose tissue segmentation result of the image to be segmented includes:
S81, determining, by using a skeletonization algorithm, a coronary artery centerline of a coronary artery foreground based on the coronary artery segmentation result.

The skeletonization algorithm is an image processing manner for extracting the coronary artery centerline from the coronary artery segmentation result. The skeletonization algorithm is used to simplify the three-dimensionally modeled coronary artery segmentation result into a single-pixel-width axis, i.e., the coronary artery centerline, so as to extract topological structures (e.g., branches and operations) and geometric features of the coronary artery. The skeletonization algorithm may be a Zhang-Suen algorithm or a Guo-Hall algorithm.

The coronary artery centerline is a single-pixel-width axis representing a central course of the coronary artery lumen. The coronary artery centerline can reflect the spatial course and anatomical locations of the coronary artery trunk and branches.

S82, for a point on the coronary artery centerline, determining a normal vector of the point based on the coronary artery centerline, a direction of the normal vector being a coronary lumen expansion direction of the coronary artery at the point, determining a distance from the point to a coronary artery lumen wall along the normal vector, and designating the distance as a coronary artery radius of the point.

In some embodiments, for a point on the coronary artery centerline, a shortest distance from the point to an outer wall of the lumen is defined as a magnitude of the normal vector, and the magnitude of the normal vector is the coronary artery radius of the point. The direction of the normal vector is a direction from the point toward the outer wall of the lumen.

S83, dilating the coronary artery such that the coronary artery radius at each point on the coronary artery centerline becomes twice the original coronary artery radius.

S84, applying a Gaussian filtering algorithm to smooth the dilated coronary artery, performing threshold segmentation on the smoothed coronary artery, and taking pixels with grayscale values within a range of [−190 HU, −30 HU] as pixels corresponding to the pericoronary adipose tissue, to obtain the adipose tissue segmentation result.

The Gaussian filtering algorithm is a noise reduction algorithm that smooths an image through convolution with a Gaussian kernel.

The grayscale value refers to an attenuation coefficient value of a pixel in a computed tomography (CT) image, used to characterize an absorption capacity of different tissues for X-rays. In pericoronary adipose tissue segmentation, because the smoothed coronary artery may include background tissue (e.g., myocardium, vascular adventitia), background tissue needs to be removed based on a difference in grayscale values between the adipose tissue and the background tissue.

In some embodiments, the adipose tissue segmentation module may retain pixels having grayscale values within a range of [−190 HU, −30 HU] as pixels corresponding to the pericoronary adipose tissue.

In some embodiments, the adipose tissue segmentation module may retain pixels having grayscale values within one of the following ranges: [−190 HU, −160 HU], [−160 HU, −130 HU], [−130 HU, −100 HU], [−100 HU, −70 HU], [−70 HU, −40 HU], [−40 HU, −30 HU], [−190 HU, −130 HU], [−160 HU, −100 HU], [−130 HU, −70 HU], [−100 HU, −40 HU], and [−70 HU, −30 HU], as pixels corresponding to the pericoronary adipose tissue.

In some embodiments, the adipose tissue segmentation module extracts the centerline from the coronary artery segmentation result using a skeletonization algorithm and performs dilation along a normal vector direction. By combining Gaussian filtering and smoothed threshold segmentation, precise localization of pericoronary adipose tissue is achieved. This process can automatically complete segmentation of the pericoronary adipose tissue without relying on a large labeled dataset, thereby helping improve efficiency of clinical evaluation of pericoronary adipose tissue (PCAT) attenuation.

The basic concepts have been described above. Obviously, for those skilled in the art, the detailed disclosure above is merely illustrative and does not constitute a limitation on the present disclosure. Although not explicitly stated, those skilled in the art may make various modifications, improvements, and corrections to the present disclosure. Such modifications, improvements, and corrections are suggested within the present disclosure and thus remain within the spirit and scope of the exemplary embodiments of the present disclosure.

Meanwhile, the present disclosure uses specific terms to describe the embodiments herein. Terms such as “one embodiment,” “an embodiment,” and/or “some embodiments” mean that a certain feature, structure, or characteristic related to at least one embodiment of the present disclosure. Therefore, it is emphasized and noted that the terms “an embodiment,” “one embodiment,” or “an alternative embodiment” mentioned two or more times at different locations in the present disclosure do not necessarily refer to the same embodiment. Furthermore, certain features, structures, or characteristics in one or more embodiments of the present disclosure may be appropriately combined.

Furthermore, unless explicitly stated in the claims, the order of processing elements and sequences, the use of numbers or letters, or the use of other names in the present disclosure is not intended to limit the order of the processes and methods described herein. Although the foregoing disclosure discusses some currently considered useful inventive embodiments through various examples, it should be understood that such details are for illustrative purposes only, and the appended claims are not limited to the disclosed embodiments. On the contrary, the claims are intended to cover all modifications and equivalent combinations that conform to the essence and scope of the embodiments of the present disclosure. For example, although the system components described above may be implemented via hardware devices, they may also be realized solely through software solutions, such as installing the described system on existing servers or mobile devices.

Similarly, it should be noted that to simplify the presentation of the present disclosure and thereby aid in understanding one or more inventive embodiments, in the descriptions of the embodiments of the present disclosure above, various features may sometimes be grouped into a single embodiment, figure, or description thereof. However, this disclosure manner does not imply that the subject matter of the present disclosure requires more features than those mentioned in the claims. In fact, the features of an embodiment are fewer than all the features of a single embodiment disclosed above.

In some embodiments, numbers describing quantities or attributes are used. It should be understood that such numbers used in the description of the embodiments may, in some examples, be modified by the terms “approximately,” “about,” or “substantially.” Unless otherwise specified, “approximately,” “about,” or “substantially” indicates that the stated number allows for a variation of +20%. Accordingly, in some embodiments, the numerical parameters used in the specification and claims are approximations, which may vary depending on the characteristics required by individual embodiments. In some embodiments, numerical parameters should consider the specified number of significant digits and adopt the method of general digit retention. Although the numerical ranges and parameters used to confirm their breadth in some embodiments of the present disclosure are approximations, in specific embodiments, such numerical values are set as accurately as possible within the feasible range.

For each patent, patent application, patent application publication, and other material, such as articles, books, specifications, publications, documents, etc., cited in the present disclosure, the entire content thereof is hereby incorporated by reference into the present disclosure. Excluded are application history documents that are inconsistent or conflict with the content of the present disclosure, and documents that limit the broadest scope of the claims of the present disclosure (whether currently or subsequently appended to the present disclosure). It should be noted that if the description, definition, and/or use of terms in the supplementary materials of the present disclosure are inconsistent or conflict with those in the present disclosure, the description, definition, and/or use of terms in the present disclosure shall prevail.

Finally, it should be understood that the embodiments described in the present disclosure are merely illustrative of the principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to those explicitly introduced and described herein.

What is claimed is:

1. A system for segmenting coronary arteries and pericoronary adipose tissue based on self-supervised learning, comprising an acquisition module, a preprocessing module, an extraction module, an encoder, a reconstruction module, a contrast module, a coronary artery segmentation module, and an adipose tissue segmentation module; wherein:
the acquisition module is configured to acquire a training set of coronary computed tomography angiography (CCTA) images, the training set including labeled images and unlabeled images;
the preprocessing module is configured to preprocess the training set and preprocess an image to be segmented;
the extraction module is configured to extract segmentation masks from the preprocessed labeled images;
the reconstruction module, the contrast module, and the encoder are configured to perform joint pre-training based on the preprocessed training set to obtain a pre-trained encoder;
the coronary artery segmentation module and the pre-trained encoder are configured to perform joint training based on the segmentation masks to obtain a trained encoder;
the encoder and the coronary artery segmentation module are configured to perform coronary artery segmentation on the preprocessed image to be segmented to obtain a coronary artery segmentation result of the image to be segmented; and
the adipose tissue segmentation module is configured to process the coronary artery segmentation result to obtain an adipose tissue segmentation result of the image to be segmented.

2. The system of claim 1, wherein the preprocessing module is further configured to:
perform cropping and standardization on the labeled images and the unlabeled images in the training set, to unify the labeled images and the unlabeled images to a same size.

3. The system of claim 2, wherein the encoder includes twelve encoding units connected in series, and the encoder is further configured to:
uniformly partition each of the preprocessed labeled images and each of the preprocessed unlabeled images into a plurality of non-overlapping patches, respectively, wherein each of the patches has a size of P×P×P, where P is a preset resolution;
flatten the partitioned patches and pass the flattened patches through a linear projection layer, then pass an output of the linear projection layer through a positional embedding layer, and perform positional embedding on the output of the linear projection layer based on the positional embedding layer to obtain positional embedding results corresponding to the patches; and
input the positional embedding results of the patches into the encoder, wherein within the encoder, the positional embedding results sequentially pass through the twelve encoding units connected in series, and feature maps output by a third encoding unit, a sixth encoding unit, a ninth encoding unit, and a twelfth encoding unit are stored to obtain a set of feature maps corresponding to each of the patches.

4. The system of claim 3, wherein each of the encoding units of the encoder includes a first normalization layer, a multi-head attention layer, a second normalization layer, and a multilayer perceptron, and the encoder is further configured to:
for any encoding unit among the encoding units of the encoder,
input an input feature of the encoding unit into the first normalization layer, and take an output of the first normalization layer as an input of the multi-head attention layer;
add an output of the multi-head attention layer and the input feature of the encoding unit to obtain a first sum result;
input the first sum result into the second normalization layer, and take an output of the second normalization layer as an input of the multilayer perceptron; and
add an output of the multilayer perceptron and the first sum result to obtain a second sum result, and take the second sum result as an output of the encoding unit.

5. The system of claim 4, wherein the reconstruction module, the contrast module, and the encoder are further configured to perform the joint pre-training by:
initializing a pre-training epoch t to 1;
flattening patches corresponding to the preprocessed training set, and sequentially processing the flattened patches through the linear projection layer, the positional embedding layer, and the encoder to obtain the set of feature maps corresponding to the patches;
for an i-th patch, denoting the patch as zᵢ, obtaining a reconstruction result \hat{z}ᵢ of the i-th patch zᵢ output from the reconstruction module by inputting the i-th patch zᵢ and the corresponding set of feature maps into the reconstruction module;
inputting the set of feature maps corresponding to the i-th patch zᵢ into the contrast module, and sequentially processing the set of feature maps corresponding to the i-th patch zᵢ through a global average pooling layer and a linear projection layer of the contrast module to obtain a feature embedding z_{i′} of the i-th patch zᵢ;
determining a total loss function L based on the reconstruction result \hat{z}ᵢ and the feature embedding zᵢ, performing backpropagation based on the total loss function L, and adjusting network parameters in an initial encoder, the reconstruction module, and the contrast module; in response to convergence of the total loss function L, pausing the backpropagation;
determining whether the pre-training epoch t has reached a maximum pre-training epoch;
in response to determining that the pre-training epoch t has not reached the maximum pre-training epoch, re-partitioning the preprocessed training set into patches, updating the pre-training epoch t to a pre-training epoch t+1, and continuing the joint pre-training for the pre-training epoch t+1;
in response to determining that the pre-training epoch t has reached the maximum pre-training epoch, completing the joint pre-training and obtain the pre-trained encoder.

6. The system of claim 5, wherein the obtaining a reconstruction result \hat{z}ᵢ of the i-th patch zᵢ further includes:
inputting the i-th patch zᵢ into a first convolutional layer, and sequentially processing the i-th patch zᵢ through the first convolutional layer, a first batch normalization (BN) layer, a first rectified linear unit (ReLU) layer, a second convolutional layer, a second BN layer, and a second ReLU layer to obtain an output a of the second ReLU layer;
sequentially processing a feature map output by a third encoding unit through a first deconvolution sub-unit, a second deconvolution sub-unit, and a third deconvolution sub-unit to obtain an output b of the third deconvolution sub-unit;
sequentially processing a feature map output by a sixth encoding unit through a fourth deconvolution sub-unit and a fifth deconvolution sub-unit to obtain an output c of the fifth deconvolution sub-unit;
processing a feature map output by a ninth encoding unit through a sixth deconvolution sub-unit to obtain an output d of the sixth deconvolution sub-unit;
processing a feature map output by a twelfth encoding unit through a first upsampling unit to obtain an output e of the first upsampling unit;
concatenating the output e and the output d, and sequentially processing a result of the concatenation through a third convolutional layer, a third BN layer, a third ReLU layer, a fourth convolutional layer, a fourth BN layer, a fourth ReLU layer, and a second upsampling unit to obtain an output i′ of the second upsampling unit;
concatenating the output c and the output i′, and sequentially processing a result of the concatenation through a fifth convolutional layer, a fifth BN layer, a fifth ReLU layer, a sixth convolutional layer, a sixth BN layer, a sixth ReLU layer, and a third upsampling unit to obtain an output j′ of the third upsampling unit;
concatenating the output b and the output j′, and sequentially processing a result of the concatenation through a seventh convolutional layer, a seventh BN layer, a seventh ReLU layer, an eighth convolutional layer, an eighth BN layer, an eighth ReLU layer, and a fourth upsampling unit to obtain an output k of the fourth upsampling unit;
concatenating the output k and the output a, and sequentially processing a result of the concatenation through a ninth convolutional layer, a ninth BN layer, a ninth ReLU layer, a tenth convolutional layer, a tenth BN layer, a tenth ReLU layer, and an eleventh convolutional layer, to obtain the reconstruction result \hat{z}ᵢ of the i-th patch zᵢ.

7. The system of claim 6, wherein the deconvolution sub-unit includes a deconvolutional layer, a convolutional layer, a BN layer, and a ReLU layer connected in series.

8. The system of claim 7, wherein
the feature embedding zᵢ′ of the i-th patch zᵢ is expressed as:
$${z}_{i}^{′} = h ( g ( f ) )$$
wherein f denotes the set of feature maps corresponding to the i-th patch zᵢ, g(f) denotes a feature vector output by the global average pooling layer, h denotes the linear projection layer, and zᵢ′ denotes the feature embedding output by the linear projection layer.

9. The system of claim 8, wherein
the total loss function L is expressed as:
$$L = {L}_{recon} + {L}_{c o n t r a s t}$$
wherein L_{recon} denotes a reconstruction loss, and L_{contrast} t denotes a contrastive loss of the patches;
$${L}_{recon} = {\sum }_{{z}_{i} ∈ M} {‖ {z}_{i} - \hat{{z}_{1}} ‖}_{2}^{2}$$
wherein, M denotes a set of all the patches, and ∥·∥₂ denotes an L2 norm;
$${L}_{c ontrast} = {\sum }_{{z}_{i} ∈ M} - log \frac{exp ( sim ( {z}_{i}^{′} , {z}^{+} ) )}{{\sum }_{{z}_{j} ∈ N ( i )} exp ( s im ( {z}_{i}^{′} , {z}_{j}^{′} ) )}$$
wherein z′ᵢ denotes the feature embedding of the i-th patch zᵢ, z⁺ denotes a feature embedding of another patch from a same CCTA image as the i-th patch zᵢ, sim(z′ᵢ, z⁺) represents a similarity between the z′ᵢ and the z⁺, N(i) represents a set of all patches other than the i-th patch zᵢ, sim(z′ᵢ, z′ⱼ) represents a similarity between the z′ᵢ and z′ⱼ, the z′ⱼ is a feature embedding of zⱼ, and the zⱼ is a j-th patch different from the i-th patch zᵢ.

10. The system of claim 9, wherein the coronary artery segmentation module and the pre-trained encoder are further configured to perform the joint training by:
initializing a training epoch p to 1;
flattening patches corresponding to the preprocessed labeled images, and sequentially processing the patches corresponding to the preprocessed labeled images through the linear projection layer, the positional embedding layer, and the encoder to obtain a set of feature maps corresponding to each of the patches;
for the i-th patch, denoting the patch zᵢ, inputting the i-th patch zᵢ and the set of feature maps corresponding to the i-th patch zᵢ into the coronary artery segmentation module;
determining a segmentation loss L′ based on the coronary artery segmentation results of the patches predicted by the coronary artery segmentation module and the segmentation masks corresponding to the patches, performing backpropagation based on the segmentation loss L′, and adjusting network parameters in the pre-trained encoder and the coronary artery segmentation module; in response to convergence of the segmentation loss L′, pausing the backpropagation;
determining whether the training epoch p has reached a maximum training epoch;
in response to determining that the training epoch p has not reached the maximum training epoch, re-partitioning the preprocessed labeled images into patches, updating the training epoch p to a training epoch p+1, and continuing the joint training for the training epoch p+1;
in response to determining that the training epoch p has reached the maximum training epoch, completing the joint training and obtaining the trained encoder.

11. The system of claim 10, wherein
the segmentation loss L′ is expressed as:
$${L}^{′} = {L}_{C E} + {L}_{D i c e}$$
wherein L_{CE} denotes a cross-entropy loss, and L_{Dice} denotes a Dice loss;
$${L}_{C E} = - {\sum }_{i = 1}^{N} {\sum }_{k = 1}^{K} {y}_{g {t}_{i , k}} log ( {y}_{p r e {d}_{i , k}} ) + ( 1 - {y}_{g {t}_{i , k}} ) log ( 1 - {y}_{p r e {d}_{i , k}} )$$
$${L}_{D i c e} = 1 - \frac{2 {\sum }_{i = 1}^{N} {\sum }_{k = 1}^{K} {y}_{g {t}_{i , k}} {y}_{{pred}_{i , k}}}{{\sum }_{i = 1}^{N} {\sum }_{k = 1}^{K} {y}_{g {t}_{i , k}} + {\sum }_{i = 1}^{N} {\sum }_{k = 1}^{K} {y}_{{pred}_{i , k}}}$$
wherein N denotes a total count of all patches corresponding to the labeled CCTA images, K denotes a count of pixels in a single patch, y_{gt}_{_{i,k}} denotes a ground-truth label for a k-th pixel from a segmentation mask corresponding to the i-th patch zᵢ, and y_{pred}_{_{i,k}} denotes a predicted result for the k-th pixel in the i-th patch zᵢ.

12. The system of claim 1, wherein to obtain the adipose tissue segmentation result of the image to be segmented, the adipose tissue segmentation module is further configured to:
determine, by using a skeletonization algorithm, a coronary artery centerline of a coronary artery foreground based on the coronary artery segmentation result;
for a point on the coronary artery centerline,
determine a normal vector of the point based on the coronary artery centerline, a direction of the normal vector being a coronary lumen expansion direction of the coronary artery at the point, determine a distance from the point to a coronary artery lumen wall along the normal vector, and designate the distance as a coronary artery radius of the point;
dilate the coronary artery such that the coronary artery radius at each point on the coronary artery centerline becomes twice the original coronary artery radius; and
apply a Gaussian filtering algorithm to smooth the dilated coronary artery, perform threshold segmentation on the smoothed coronary artery, and take pixels with grayscale values within a range of [−190 HU, −30 HU] as pixels corresponding to the pericoronary adipose tissue, to obtain the adipose tissue segmentation result.
